(19) United States
(12) Patent Application Publication
(10) Pub. No.: US 2007/0270894 A1
(43) Pub. Date: Nov. 22, 2007

(54) Combined endoscope and biopsy instrument with a removable biopsy cassette for in situ fixation and specimen processing

(76) Inventor: David S. Zimmon, Port Washington, NY (US)

Correspondence Address: WILLIAM COLLARD;COLLARD & ROE, P.C., 1077 NORTHERN BOULEVARD, ROSLYN, NY, 11576, US

(21) Appl. No.: 11/435,058

(22) Filed: May 16, 2006

(51) Int. Cl. A61B 17/32

(52) U.S. Cl. 606/170

(57) ABSTRACT

A combined endoscope and biopsy device with either a distal separable or replaceable biopsy cassette or a distal removable replaceable internal biopsy cassette with orientation, serial collection, storage and processing of biopsy specimens in situ when used with a rigid or flexible endoscope of any size. Similarly, the motive force of suction or fluid pressure may be used to propel the biopsy from the distal mechanism that cuts and captures the biopsy into a proximal collection cassette at the proximal end of the endoscope or biopsy instrument for immediate processing and analysis without removing and destroying the biopsy instrument.

### BACKGROUND OF THE INVENTION

1. Field of the Invention

The present invention relates to an endoscope combined with an apparatus for tissue acquisition with orientation, serial collection, storage and processing of the acquired specimens in situ. The device cuts and captures biopsy specimens of a closely defined size to permit serial entry into a separable or removable distal storage cassette, a removable distal internal cassette or a proximal collection or removable cassette for transportation, or in situ chemical, biological or genetic testing by immediately reacting the biopsy specimens before metabolic changes, degradation or contamination can occur or for fixation, staining and other processing or analysis. The cassette may be optically transparent for physical analysis of the tissue without removal from the cassette after separation from the biopsy instrument and endoscope. Prior to biopsy, an open tube shaft with a side arm permits fluid sampling, irrigation, and injection of tissue stains or radiopaque contrast agents.

2. The Prior Art

It is often necessary to acquire tissue samples for examination from deep within structures. These samples can only be retrieved by catheterization methods using endoscopic or fluoroscopic control. The biopsy devices previously used for these techniques were passed through an instrument channel in the endoscope to remove 1 to 4 specimens that were retrieved by removing the biopsy instrument from the endoscope and patient, and placing the specimen in a container of fixative solution labeled with the biopsy site and patient identification. During this process of acquisition and collection, minute specimens were frequently lost and were always contaminated by exposure to adjacent tissue fluids, passage through the endoscope instrument channel and handling. The need for two devices, namely the endoscope and biopsy instrument, required the fabrication and manipulation of two instruments. This necessitated the coordinated action of two trained operators and the cost of two precision instruments. Furthermore during acquisition and handling, the staff is exposed to potentially infectious tissue and toxic fixatives.

The biopsies obtained at each biopsy site and pass were processed in a batch, since the minute pieces can not be easily separated. Multiple biopsy passes were required because of the limited storage capacity of the biopsy instruments and the need to identify the origin of each biopsy site. After each biopsy pass the biopsy(s) must be removed from the biopsy instrument and placed in a labeled fixative container. The biopsy instrument washed to remove fixative and returned to the endoscopist for passage through the endoscope channel for the next biopsy. Consequently, considerable effort and expense was required to handle biopsies from different anatomic sites separately. The procedure could fail if the position of the endoscope or biopsy instrument could not be reacquired during the repeated passes through the endoscope. This complexity prolonged the endoscopic procedure and increased the quantity of sedative administered to the patient, risk and cost.

The containers for each patient were then transported to the pathology laboratory where each container was opened and each specimen batch transferred to individual numbered cassettes that were recorded for later identification. The cassettes were then processed for examination. The processed specimen batches were then sliced, stained and mounted on individual labeled slides for microscopic examination. The specimens in each container must be processed and mounted on slides separately to maintain identification. This was particularly important to prevent errors in reporting and when the distribution and extent of a cancer was being mapped to determine therapy.

During this complex handling process, small specimens may be lost or damaged. At each stage of handling, the staff is exposed to infection from the biopsies and toxic fixative. This is particularly true when the unfixed specimen is removed from the sharp biopsy instrument before fixation. The staff is also exposed to solvent vapor from the fixative at each transfer step. The solvents may be allergenic or carcinogenic. This tedious, labor intensive process is expensive in staff required, time, equipment and laboratory space.

Specimens needed for chemical, biological or genetic testing require additional biopsies that must be handled separately. These specimens were contaminated by issue fluid and material within the channel of the endoscopic instrument. The delay in acquisition and contamination of the specimens limited the accuracy and reliability of the subsequent analysis. This disability may be severe when genetic or other biological testing is required.

The prior art described in the spring based multi-purpose medical instrument in U.S. Pat. No. 5,782,747 to Zimmon, the disclosure of which is herein incorporated by reference, obviates the use of cumbersome metal shafts and coverings that occupy the space needed for specimen storage within the biopsy instrument. Prior jaw-fulcrum biopsy devices require a stiff shaft to prevent kinking and binding within the endoscope when the actuator cable(s) is pulled to close the biopsy jaws and then held to maintain jaw closure when removing the device and biopsy from the endoscope or access passage. The combined stiffness of the shaft and pull on the actuator cable(s) straightens the biopsy device and endoscope. This action moves the endoscope and biopsy device away from the biopsy site, limits maneuverability and prevents rapid serial biopsy of the target site. This stiffness and uncontrolled motion also risks trauma to the biopsy site and limits access in curved lumens. A further limitation of stiff shafts is that they reduce carrier instrument (endoscope) flexibility and maneuverability.

The closing force of a traditional jaw-fulcrum forceps biopsy instrument is limited by a shaft length ranging from 100 cm to 220 cm and the multiple curves traversed within the endoscope that must conform to a lumen. Because of these disabilities, endoscopic jaw-fulcrum biopsy forceps rip the mucosal biopsy from the muscularis mucosa. This gives a biopsy that is larger than the biopsy instrument shaft diameter and varies in size. Furthermore, tissue distortion from biopsy trauma makes histopathologic interpretation complex, because of crush and shear artifacts. These artifacts may result in false positive and false negative histopathologic interpretations of biopsy specimens, leading to an incorrect diagnosis and the need to repeat the endoscopic biopsy procedure.

U.S. Pat. Nos. 5,685,320 and 5,782,747, both to Zimmon, both of which are herein incorporated by reference, solve these problems by sharply cutting biopsies of defined size that are suitable for collection in a storage cassette.

In U.S. Pat. No. 5,685,320, the spring based multi-purpose medical instrument compresses folded spring sharp biopsy cups by sliding the tube shaft over a folded spring. The actuator wire holds the folded spring blade in position during biopsy cutting. The closing biopsy cups both cut and capture a biopsy of controlled size that is captured by the storage cassette.

The motive force of suction or fluid pressure may be used to propel the precisely cut biopsy into a proximal collection cassette as described in U.S. Pat. No. 6,071,248 to Zimmon, which is herein incorporated by reference.

In U.S. Pat. No. 6,322,522 to Zimmon, which is herein incorporated by reference, the spring based multi-purpose medical instrument is modified to capture biopsy specimens in a removable cassette or cassettes at the proximal end of the biopsy instrument for immediate processing and analysis without removing and destroying the biopsy instrument to form the cassette. This improvement allows real time specimen analysis during the biopsy procedure and the use of a relatively expensive reusable or reprocessable biopsy instrument.

In U.S. Pat. Nos. 5,980,468 and 6,071,248 to Zimmon, which are herein incorporated by reference, the orientation, serial collection, storage and processing of multiple specimens within a biopsy instrument cassette yields a great savings of time and effort in processing the biopsies, as well as preventing specimen loss or damage during handling and protecting staff from infectious material and toxic fixatives. This goal is facilitated by minimizing the operating parts of the biopsy instrument to maximize the storage volume.

In Published United States Patent Application No. 2005/0256424 to Zimmon, which is herein incorporated by reference, the separable distal cassette is modified to hold a replaceable internal cassette.

Although the prior art has made safe efficient biopsy deep within the patient possible, the need for additional improvements remains.

### SUMMARY OF THE INVENTION

It is therefore an object of the present invention to provide a combined endoscope and biopsy device with either a distal separable or replaceable biopsy cassette, or a distal removable replaceable internal biopsy cassette with orientation, serial collection, storage and processing of biopsy specimens in situ when used with a rigid or flexible endoscope of any size. Similarly, the motive force of suction or fluid pressure may be used to propel the biopsy from the distal mechanism that cuts and captures the biopsy into a proximal collection or removable cassette or cassettes at the proximal end of the endoscope or biopsy instrument for immediate processing and analysis without removing and destroying the biopsy instrument.

The biopsy instrument may be mated with the endoscope in a number of different ways. Each method has specific advantages:

(1) In a preferred device according to the invention, the entire biopsy mechanism is fully incorporated into the endoscope with the biopsy actuator in the endoscope handle, and only the actuator wire passing down the endoscope shaft to a separable or removable distal biopsy cassette in the distal endoscope section.

(2) In another embodiment of the device according to the invention, the biopsy actuator is in the endoscope handle and the actuator wire passes down the endoscope shaft within a dedicated instrument channel to a separable or removable distal biopsy cassette within the distal endoscope section. There is a side arm connected to the instrument channel for injection or aspiration.

(3) In a further embodiment according to the invention, the biopsy actuator is in the endoscope handle and the actuator wire passes down the endoscope shaft to exit to an externally placed separable or removable distal biopsy cassette outside the endoscope or attached to the endoscope cover.

(4) In yet another embodiment according to the invention, the removable and replaceable proximal storage cassette is separable from the distal removal biopsy cutting and capture mechanism, either external to the endoscope or connected through a dedicated endoscope instrument channel for immediate external collection of biopsy specimens.

(5) In another embodiment according to the invention, the biopsy apparatus is incorporated into the distal part of the endoscope and the shaft, actuator wire and handle are outside of the endoscope.

(6) In another embodiment of the device according to the invention, the entire biopsy instrument is on the outside and attached to the endoscope or is only attached to the distal end of the endoscope.

(7) In another embodiment of the device according to the invention the biopsy actuator is in the endoscope handle and operates a remote distal internal or externally placed removable replaceable or internal biopsy cassette by an electrical, radio and other signal that activates a solenoid to operate the remote biopsy mechanism directly or through a short actuator wire.

The advantages of external partial connection of the biopsy instrument to the endoscope are improved biopsy acquisition without modification of existing endoscopes. This is particularly useful when the endoscope diameter is narrow and endoscope modification would be difficult and expensive.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a longitudinal view of one embodiment of the device according to the invention fully incorporated into the endoscope with only the actuator wire passing down the endoscope shaft;

FIG. 2 shows a longitudinal view of another embodiment of the device according to the invention with the biopsy instrument incorporated within a dedicated biopsy instrument channel with a side arm for injection and aspiration;

FIG. 3 shows a longitudinal view of a partial section of another embodiment of the device according to the invention with the biopsy instrument actuator wire and control incorporated into the endoscope with the replaceable or removable biopsy cassette attached to the outside of the distal most part of the endoscope;

FIG. 4 shows a longitudinal view of yet another embodiment of the device according to the invention with the entire biopsy device incorporated within a dedicated biopsy instrument channel with a proximal replaceable cassette;

FIG. 5 shows a longitudinal view of another embodiment of the device according to the invention with the biopsy cassette incorporated into the distal part of the endoscope with the biopsy device shaft, actuator wire and handle outside of the endoscope;

FIG. 6 shows a longitudinal view of a partial section of another embodiment of the device according to the invention with the biopsy actuator handle and shaft of the biopsy instrument separated from the endoscope with the distal biopsy cassette attached externally to the distal section of the endoscope; and

FIG. 7 shows a longitudinal view of a further embodiment of the device according to the invention with the biopsy actuator in the endoscope handle that operates a remote distal biopsy cassette by a signal that activates a solenoid to operate the remote biopsy mechanism directly or through a short actuator wire.

### DETAILED DESCRIPTION OF THE PREFERRED EMBODIMENT

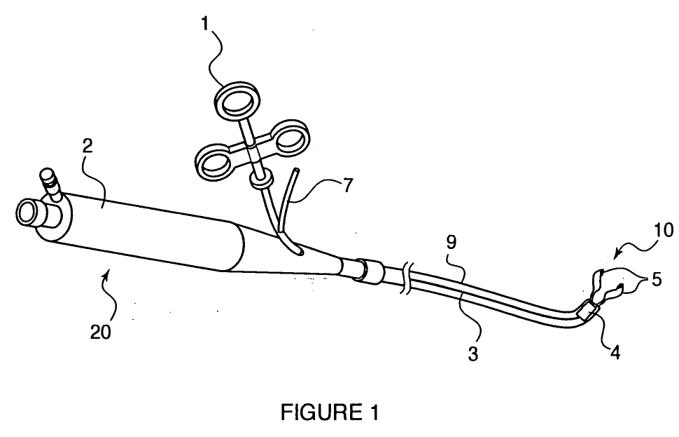

FIG. 1 shows a longitudinal view with the entire biopsy 10 device incorporated within the endoscope 20. The actuator 1 is positioned within endoscope handle 2 and only fine actuator wire(s) 3 passes down the endoscope shaft 9 to the distal biopsy cassette 4 in the distal section of the endoscope.

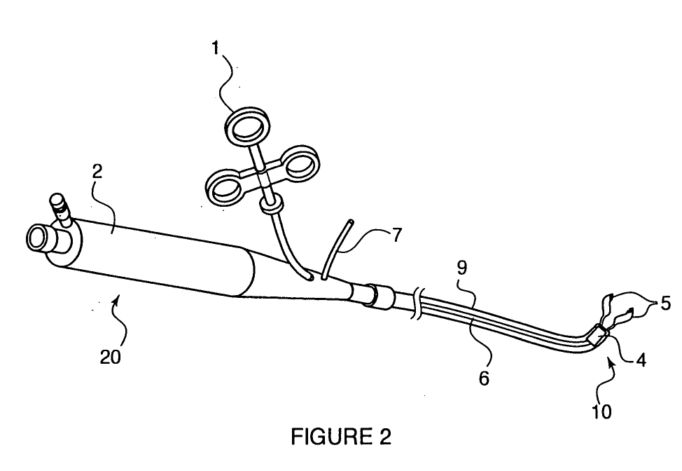

FIG. 2 shows a longitudinal view with the biopsy instrument incorporated within a dedicated biopsy instrument channel 6 of the endoscope 20 with a side arm 7 for aspiration or irrigation through the channel 6. Actuator 1 is positioned within or outside the endoscope handle 2 and actuator wire(s) 3 passes down endoscope channel 6 to distal biopsy cassette 4 incorporated into a distal section of the endoscope. Sidearm 7 connected to the channel either within the endoscope or through the outside handle permits irrigation or aspiration through the channel.

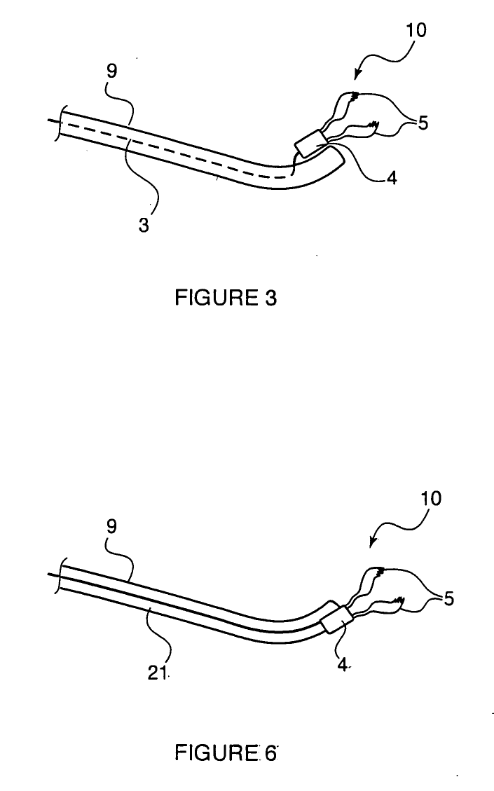

FIG. 3 shows a longitudinal view with actuator 1 in endoscope handle 2 and actuator wire 3 incorporated into the endoscope shaft with the actuator wire exiting to the distal biopsy cassette 8 attached to the outside of the distal section of the endoscope.

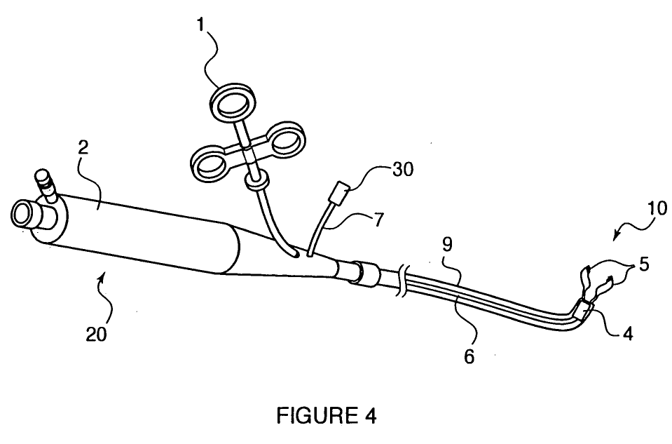

FIG. 4 shows a longitudinal view with the biopsy instrument incorporated within a dedicated biopsy instrument channel 6 of the endoscope 20 with a side arm 7 for aspiration or irrigation through dedicated channel 6 with a proximal replaceable cassette 30.

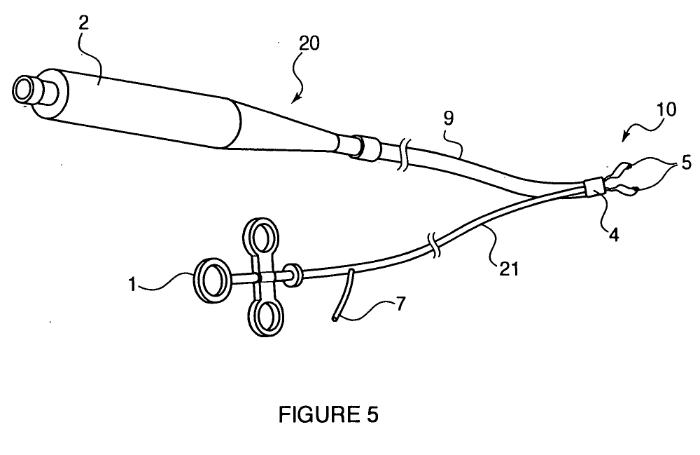

FIG. 5 shows a longitudinal view with only biopsy cassette 4 incorporated into the endoscope distal section, with the biopsy instrument actuator handle 1 and shaft 21 containing actuator wire 3 and sidearm 7 separated from and outside the endoscope 20.

FIG. 6 shows a longitudinal view with the entire biopsy instrument 10 attached to the outside the endoscope 20. The attachment may be fixed or removable. This preferred embodiment has the advantages of improved biopsy acquisition without modification of existing endoscopes and is particularly useful when the endoscope is rigid or diameter is narrow, where endoscope modification would be difficult and expensive.

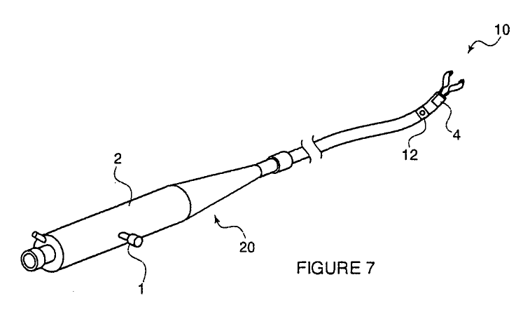

FIG. 7 shows a longitudinal view with a remote actuator 11 in endoscope handle 2 that remotely operates a solenoid 12 by a signal to directly or through a short actuator wire operate distal biopsy cassette 4 placed within the distal endoscope section or attached to the outside of the distal section of the endoscope 20.

Accordingly, while only a few embodiments of the present invention have been shown and described, it is obvious that many changes and modifications may be made thereunto without departing from the spirit and scope of the invention.

What is claimed is:

1. An apparatus for performing a medical procedure, comprising:
an endoscope having a handle and a shaft;
a biopsy device having a removable or replaceable biopsy cassette or a replaceable internal biopsy cassette, said biopsy device being connected with the endoscope; and
an actuating mechanism for the biopsy device, said actuating mechanism being incorporated into the handle of the endoscope and passing through the endoscope shaft to the biopsy device.

2. The apparatus of claim 1, wherein the biopsy device is incorporated in a channel within the endoscope, said channel having a proximal side arm for injection and aspiration through the channel.

3. The apparatus of claim 1, wherein the biopsy cassette is attached to an outside of the endoscope and wherein the actuator passes inside the endoscope to the biopsy cassette.

4. The apparatus of claim 1, wherein the entire biopsy device is incorporated in a channel within the endoscope and further comprising a proximal removable storage cassette connected to the biopsy device.

5. The apparatus of claim 1, wherein the biopsy cassette is incorporated into a distal part of the endoscope, and wherein a shaft of the biopsy device, the actuator and the handle are all disposed outside of the endoscope.

6. The apparatus of claim 1, wherein the entire biopsy device including the cassette is attached to an external cover or to the shaft of the endoscope.

7. The apparatus according to claim 1, wherein the biopsy device further comprises a removable proximal storage cassette and wherein the storage cassette and the biopsy cassette are attached externally to the endoscope.

8. The apparatus according to claim 1, wherein the biopsy device further comprises a removable proximal storage cassette and wherein the storage cassette and the biopsy cassette are attached through a dedicated endoscope instrument channel.

9. The apparatus according to claim 1, wherein the distal biopsy device is remotely activated by an actuator in the endoscope handle through electrical, radio or other signal.
